(19) United States
(12) Patent Application Publication
(10) Pub. No.: US 2025/0084466 A1
(43) Pub. Date: Mar. 13, 2025

(54) DEVICES, KITS, AND METHODS FOR DETERMINING INCREASED SUSCEPTIBILITY TO AND TREATMENT AND PREVENTION OF PERIODONTITIS, ALZHEIMER’S DISEASE, AND OTHER CONDITIONS

(71) Applicants: Martin Levine, Norman, OK (US); Semmelweis Egyetem, Budapest (HU)

(72) Inventors: Martin Levine, Norman, OK (US); Zsolt M. Lohinai, Budapest (HU); Anna Zsuzsanna FÖldes, Budapest (HU)

(21) Appl. No.: 18/958,170

(22) Filed: Nov. 25, 2024

Related U.S. Application Data
(63) Continuation-in-part of application No. 18/577,460, filed on Jan. 8, 2024.
(60) Provisional application No. 63/219,622, filed on Jul. 8, 2021.

(51) Int. Cl. C12Q 1/6827; A61K 33/16; A61K 33/30; B01L 3/00; C12Q 1/6883

(52) U.S. Cl. CPC C12Q 1/6827; A61K 33/16; A61K 33/30; B01L 3/502715; C12Q 1/6883; C12Q 2600/106; C12Q 2600/156

(57) ABSTRACT

Diagnostic microarray devices, kits, and methods of treating or reducing the occurrence of various conditions or diseases are disclosed, wherein the conditions or diseases include (but are not limited to) periodontal disease, Alzheimer's disease, cardiovascular disease, arthritis, and adverse pregnancy outcomes. The devices, kits, and methods utilize an analysis of single nucleotide polymorphisms (SNPs) from various interleukins.

### CROSS REFERENCE TO RELATED APPLICATIONS/INCORPORATION BY REFERENCE STATEMENT

This application is a continuation-in-part of U.S. Ser. No. 18/577,460, filed Jan. 8, 2024; which is a US national stage application filed under 35 USC § 371 of International Application No. PCT/US2022/033045, filed Jun. 10, 2022; which claims benefit under 35 USC § 119(e) of U.S. Provisional Application No. 63/219,622, filed Jul. 8, 2021. The entire contents of the above-referenced patent applications are hereby expressly incorporated herein by reference.

### STATEMENT REGARDING FEDERALLY SPONSORED RESEARCH OR DEVELOPMENT

Not Applicable.

### BACKGROUND

Rapid advances in technology are bringing medicine close to tailoring genetic information into therapies for optimal health. Periodontal disease (PD), an inflammatory disease that destroys tooth attachments, is mediated by dentally adherent bacterial biofilms. Mastication produces traces of an inflammatory exudate called gingival crevicular fluid (GCF), and this fluid transudes into crevices between the teeth and gingiva where it provides nutrients for the basal layer of the dental epithelial attachment so that it maintains a proliferative phenotype and behaves as a barrier to dentally adherent bacteria. The saliva of most young adults contains traces of periodontopathic bacteria picked up from their social environment. These bacteria include a keystone pathogen, Porphorymonas gingivalis, along with Tannerella forsythia, Treponema denticola, and some others. These strictly anaerobic, protease-dependent bacteria, commonly known as the red complex, increase when the salivary biofilm is incubated in a medium simulating GCF in vitro, or when oral hygiene is inadequate, and GCF exudation remains increased in vivo.

The red complex normally takes many years to become established within dental biofilms, but once it does, the keystone bacterium, P. gingivalis, is unique in possessing a group of powerful secreted proteases called gingipains (gp) that can destroy the periodontium. Gingipains are divided into two major groups depending on whether they cleave after lysine (K) or arginine (R) residues in proteins (Kgp, or Rgp). These and other proteases in the red complex degrade collagen, fibronectin, various cytokines that disturb the host cytokine network, host defense mechanisms, interactions between host cells, the extracellular matrix, and the viability of fibroblasts and endothelial cells. The gingipain hypothesis proposes that the destruction of the periodontium allows P. gingivalis to break into the local systemic blood flow and spread to many different parts of the body, including the brain.

P. gingivalis is not normally detectable in the oral cavity prior to age 13, and this bacterium first appears with other red bacteria in young adults (>age 19), especially where pocket depth exceeds 2.0 mm. Dentally adherent bacteria are mostly viridans streptococci and actinomyces species, but other bacteria normally present in saliva appear within a week of discontinuing oral hygiene. One of these other bacteria is E. corrodens which produces lysine decarboxylase (LdcE), an enzyme, that depletes the GCF of its lysine by converting it to cadaverine and carbon dioxide. Lysine is an essential amino acid, and the reduced lysine content of GCF impairs the epithelial barrier to bacteria at the crevice base. The epithelial attachment becomes exposed to microbial products that interact with receptors in the host epithelium to enhance GCF exudation, and the bacterial composition starts changing 2-3 weeks of discontinued oral hygiene.

The red complex develops through many intermediate stages as gingival inflammation (gingivitis) persists, and its component bacteria catabolize enough GCF proteins to precipitate calcium phosphate around biofilm bacteria (dental calculus). The calcified attachment to teeth surfaces requires assistance to remove the calculus and underlying biofilm. Otherwise, gingival margins start to recede (periodontitis), cementum becomes exposed in the oral cavity (moderate periodontitis), and eventually the teeth may loosen and exfoliate, or require to be removed (severe periodontitis). Surprisingly, moderate and severe periodontitis develop in only about 38% of the population aged 30 years and older. In these individuals, periodontitis associates with common chronic inflammatory diseases such as cardiovascular diseases, rheumatoid arthritis, and Alzheimer's disease (AD). Although cardiovascular and rheumatoid arthritis can be diagnosed and controlled, periodontal and Alzheimer's diseases are neither predictable nor easily treated. Dental implants or dentures are options for treating moderate or severe periodontal disease, but only palliative therapies are available for Alzheimer's disease. At present, a major consideration revolves around studies indicating that P. gingivalis and its gingipain products in the brain promote Alzheimer's disease (Nara, et al., Journal of Alzheimer's Disease: JAD (2021) 82(4):1417-1450).

Studies of twins and epidemiological investigations of the natural history of periodontitis suggest that genetics play an important role in determining an individual's clinical presentation. The pro-inflammatory cytokine interleukin-1 (IL1) is a key regulator of host inflammatory responses to microbial infections. There are two IL1 genes, IL1A and IL1B. The former synthesizes protein IL-1α, and the latter synthesizes protein IL-1β. The respective amino acid sequences are only 34% homologous. IL-1α is constantly expressed within cells but only secreted after intrinsic or bacterial damage by bacteria or other foreign agents contacting the cell surface or invading the cytosol. These processes activate innate immunity which releases IL1α into the interstitial fluid. IL1α then binds to a receptor on surrounding cells through which it activates the synthesis and secretion of IL1β. from the surrounding epithelial, fibroblast, and other cells of the gingiva. The conventional wisdom is that the longer the initially adherent dental plaque (biofilm) remains, the more IL13 is secreted, and the greater is gingival inflammation (gingivitis) and the faster is the development of the ‘red’ complex. and its downstream cytokines are activated by microbial products.

A single nucleotide polymorphism (SNP) upstream of the protein encoding site at residue 889 (IL1A-889) increases IL-1α expression 3-fold, and associates with severe periodontitis (Kiani, et al., Iran J Allergy Asthma Immunol (2009) 8(2):95-98). Unlike IL-1α, IL-1β appears only after IL-1α is activated and released into the interstitial fluid, in the presence of microbial products. The IL1B gene can possess an SNP at residue +3877 in an intron that interacts with some or all of 3 SNPs in the IL1B promoter region (−511, −1464, and −3737 upstream from the protein encoding region). Despite the possibility of many such potential interactions, only 5 such interactions associate with periodontitis, of which one was strongly and significantly detected in multiple ethnicities residing in the US and elsewhere (Wu, X., et al., J. Periodontal Res (2015) 50(1):52-61).

In addition, a nearby SNP at ILB+3954 (77 nucleotides downstream from IL1B+3887), lies within the IL1β polypeptide-encoding region without changing the encoded amino acid sequence. Greater levels of this other IL-1β protein possessing this SNP IL1B+3954 are related to the presence of the red microbial complex and periodontal tissue destruction (Ferreira, et al., Infect Immun (2008) 76(8):3725-3734). Three independent predictors of a severe disease phenotype (tooth-loss) are recognized: a) smoking tobacco; b) type 2 (adult onset) diabetes; and c) an IL1A-889(GA)/IL1B+3954(GA) genotype. The specified (major) nucleotide for both IL1A-889 (rs1800587) and IL1B+3954 (rs1143634) is guanine (G) and the alternate (minor) nucleotide is adenine (A). Strand orientation of the DNA double helix indicates that the IL1B+3954 major/minor nucleotides are not C/T as reported by Giannobile, et al., J Dent. Res (2013) 92(8):694-701. The genotype IL1A-889(A)/IL1B+3954(A) is present in over 60% of smokers, but only in 7% of the US population. This estimate derived from the minor (alternate) SNP frequencies, in both genes validated in a large European population (Karimbux et al., J Periodontol (2012):83:(11); 1407-1419). Nevertheless, moderate plus severe cases of periodontitis account for 38% of individuals aged 30 through 65 years in the US (Genco and Borgnakke, Periodontol 2000 (2013):62(1); 59-94). The tooth-loss genotype, IL1A-889(A)/IL1B+3954(A), does possess not a broad enough clinical phenotype to indicate differences in disease susceptibility. Such a phenotype should be capable of predicting the 38% of individuals susceptible to developing P. gingivalis in deepened crevices associated with periodontitis. This bacterium has powerful proteases that may cause other diseases and conditions, such as (but not limited to) late onset Alzheimer's and vascular dementia known to associate with periodontitis (Guo et al., Int. J. Environ. Res. Public. Health (2021):18(13); 6823).

Therefore, there is a need in the art for new and improved devices, kits, and methods for prediction, identification, diagnosis, and treatment of various disorders associated with severe periodontitis. It is to such devices, kits, and methods, which utilize a phenotype for a weak inflammatory response to periodontitis, that the present disclosure is directed.

### BRIEF DESCRIPTION OF THE DRAWINGS

FIG. 1 graphically illustrates the separation of host genotypes for gingival exudation rate phenotypes into two clusters. The upper curve indicates strong GCF responders to lysine in their biofilm, and the lower curve indicates weak GCF responders (the circle with a small gray spot above the lower of the two curves indicates a participant unavailable for genotyping). Unfilled circles indicate nine participants assigned only by different combinations of IL1B-511 and IL1B+3877 alleles. The phenotypes of another two participants were changed by epistasis (i.e., reversal of the normal phenotype expression) in the presence of the T allele of IL6-1363, and have a central dark spot. The association of these 11 participants with the appropriate response gave a Fisher exact test statistic=0.007, p<0.01. The circles designated 1 through 4 indicate participants possessing both IL1B-511(A) and IL1B+3877(T) alleles, a confounding genotype that required a second gene's SNP, indicated by specifies (s) in FIG. 1, to determine their GCF response. Host #2 indicated as s* had two specifying SNPs. Weak responders 1 and 2 on FIG. 1 had different separation genes from the two strong responders numbered 3 and 4 in the upper curve. Surprisingly, none of the 15 participants possessed the common allele at both IL1B-511 and IL1B+3877.

FIG. 2 contains genotypes that identify the strong (S) and weak (W) GCF responses shown in FIG. 1. By convention, the gene name is italicized, and the site of a single polymorphism location is indicated by the number of nucleotides upstream (−) or downstream (+) from the translational start site of the encoded polypeptide. The National Center for Biotechnology Information (NCBI) has a Reference SNP (rs) portal that provides an ID number beginning with ‘rs’ for all known SNPs. The reference or common nucleotide is named first, A=Adenine, G=Guanine, C=Cytidine, or T=Thymidine, and the alternate nucleotide is named following a >sign to indicate that it is the minor allele frequency (MAF) for that site. The MAF in European or other large populations is also available. Hosts are identified by row numbers in lane i. Lanes iia and iib contain the SNPs that identify hosts whose genome possesses a long-studied genotype associated with severe periodontitis and tooth loss. Lanes iii through ix identify other SNPs from genes known to associate with moderate or severe periodontitis and were related to strong or weak GCF in this study. The reference SNP allele of each gene is shown first, and if the SNP alternate allele is also present (heterozygous), the background of the nucleotide is colored green. Otherwise, if both copies on the chromosome contain the reference SNP (homozygous) and the gene is translated from the forward (5′) DNA strand, the reference SNP is colored red and the alternate homozygous SNP is colored blue as in lane iii. If the reference SNP is translated from the complementary (3′ strand), the homozygous box is colored blue, and the homozygous alternate SNP box is colored red as in lane iv. SNPs in boxes with a white background are unrelated to the study. Epistasis^{e} and its dominanced indicate presence of the alternate T SNP at IL6-1363 in hosts from rows 1 and 11. Specifies^{s} indicates a second gene's SNP required to differentiate hosts possessing a ‘conflicted’ genotype based on the relationship of groups of SNPs to strong or weak GCF response. Square brackets around a gene name and location bearing the symbol ‘s*’ (only present in host 3) indicates an SNP from an additional gene that may specify the same strong or weak GCF response.

FIG. 3 contains a complete list of the SNP alleles in the genes whose alleles were investigated herein along with their relationship to SNP alleles of the tooth loss genotype. Column i identifies each participant (Host). Columns ia, ib, ic, and id indicate sex, lysine contents, GCF exudation rates, and PI values, respectively. Column ii indicates the Strong or Weak (S/W) phenotype. Columns iia and iib indicate the genes of the tooth loss phenotype IL1A-889 and IL1B+3954 as in FIG. 2, and Columns iii-viii indicate the alleles of IL1B+3877, IL1B-511, IL6-1363, IL10-597, CD14-260, and COX2+8473, respectively. The ninth allele (Column ix) was the MMP8-799 gene; this gene did not appear to associate with either strong or weak phenotype in this Example. Columns left to right are numbered as in FIG. 2, but here the white boxes are filled in to display allelic variants not associated with strong or weak response.

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions and/or methods disclosed herein can be made and executed without undue experimentation in light of the present disclosure. While the compositions and methods of the inventive concept(s) have been described in terms of particular embodiments, it will be apparent to those of skill in the art that variations may be applied to the compositions and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the phrases “associated with” and “coupled to” include both direct association/binding of two moieties to one another as well as indirect association/binding of two moieties to one another. Non-limiting examples of associations/couplings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

The term “pharmaceutically acceptable” refers to compounds and compositions which are suitable for administration to humans and/or animals without undue adverse side effects such as (but not limited to) toxicity, irritation, and/or allergic response commensurate with a reasonable benefit/risk ratio.

The term “pharmaceutically-acceptable excipient” refers to any carrier, vehicle, and/or diluent known in the art or otherwise contemplated herein that may improve solubility, deliverability, dispersion, stability, and/or conformational integrity of the compositions disclosed herein.

The term “patient” as used herein includes human and veterinary subjects. “Mammal” for purposes of treatment refers to any animal classified as a mammal, including (but not limited to) humans, domestic and farm animals, nonhuman primates, and any other animal that has mammary tissue.

The term “treatment” refers to both therapeutic treatment and prophylactic or preventative measures. Those in need of treatment include, but are not limited to, individuals already having a particular condition/disease/infection as well as individuals who are at risk of acquiring a particular condition/disease/infection (e.g., those needing prophylactic/preventative measures). The term “treating” refers to administering an agent/element/method to a patient for therapeutic and/or prophylactic/preventative purposes.

A “therapeutic composition” or “pharmaceutical composition” refers to an agent that may be administered in vivo to bring about a therapeutic and/or prophylactic/preventative effect.

Administering a therapeutically effective amount or prophylactically effective amount is intended to provide a therapeutic benefit in the treatment, prevention, and/or management of a disease, condition, and/or infection. The specific amount that is therapeutically effective can be readily determined by the ordinary medical practitioner, and can vary depending on factors known in the art, such as (but not limited to) the type of condition/disease/infection, the patient's history and age, the stage of the condition/disease/infection, and the co-administration of other agents.

The term “effective amount” refers to an amount of a biologically active molecule or conjugate or derivative thereof, or an amount of a treatment protocol (i.e., an alternating electric field), sufficient to exhibit a detectable therapeutic effect without undue adverse side effects (such as (but not limited to) toxicity, irritation, and allergic response) commensurate with a reasonable benefit/risk ratio when used in the manner of the inventive concept(s). The therapeutic effect may include, for example but not by way of limitation, preventing, inhibiting, or reducing the occurrence of at least one tumor and/or cancer. The effective amount for a subject will depend upon the type of subject, the subject's size and health, the nature and severity of the condition/disease/infection to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

As used herein, the term “concurrent therapy” is used interchangeably with the terms “combination therapy” and “adjunct therapy,” and will be understood to mean that the patient in need of treatment is treated or given another drug for the condition/disease/infection in conjunction with the treatments of the present disclosure. This concurrent therapy can be sequential therapy, where the patient is treated first with one treatment protocol/pharmaceutical composition and then the other treatment protocol/pharmaceutical composition, or the two treatment protocols/pharmaceutical compositions are given simultaneously.

The terms “administration” and “administering,” as used herein, will be understood to include all routes of administration known in the art, including but not limited to, oral, topical, transdermal, parenteral, subcutaneous, intranasal, mucosal, intramuscular, intraperitoneal, intravitreal, and intravenous routes, and including both local and systemic applications. In addition, the compositions of the present disclosure (and/or the methods of administration of same) may be designed to provide delayed, controlled, or sustained release using formulation techniques which are well known in the art.

Turning now to the inventive concept(s), certain non-limiting embodiments of the present disclosure are directed to diagnostic microarray devices, kits, and methods for use in determining increased susceptibility to and treatment and prevention of periodontitis as well as other systemic conditions that have been shown to be associated therewith, such as (but not limited to) Alzheimer's Disease, cardiovascular disease, arthritis, adverse pregnancy outcomes, and others.

Certain non-limiting embodiments are directed to a diagnostic microarray device that includes a substrate formed of a glass material and a plurality of probes attached or otherwise associated with the substrate. Each probe is associated with the substrate at a defined location that is spatially separated from the other probes so as to allow for detection of a signal generated by each probe. Each probe is specific to one allele of a single nucleotide polymorphism (SNP).

In certain non-limiting embodiments, the probes comprise one or more of the following: a probe that detects a G allele of IL1A-889; a probe that detects an A allele of IL1A-889; a probe that detects a C allele of IL1B+3877; a probe that detects a T allele of IL1B+3877; a probe that detects a G allele of IL1B-511; a probe that detects an A allele of IL1B-511; a probe that detects a G allele of IL6-1363; a probe that detects a T allele of IL6-1363; a probe that detects a G allele of IL10-597; a probe that detects a T allele of IL10-597; a probe that detects a G allele of CD14-260; a probe that detects an A allele of CD14-260; a probe that detects an A allele of COX2+8473; a probe that detects a G allele of COX2+8473; a probe that detects an A allele of MMP8-799; a probe that detects a G allele of MMP8-799; a probe that detects a C allele of IL10-1082; a probe that detects a T allele of IL10-1082; and/or a probe that detects a TREM2 R47H mutation.

In certain particular (but non-limiting) embodiments, the probes comprise two or more of the above, three or more of the above, four or more of the above, five or more of the above, six or more of the above, seven or more of the above, eight or more of the above, nine or more of the above, 10 or more of the above, 11 or more of the above, 12 or more of the above, 13 or more of the above, 14 or more of the above, 15 or more of the above, 16 or more of the above, 17 or more of the above, 18 or more of the above, or all 19 of the above probes.

The diagnostic microarray device can be utilized for detecting susceptibility to (for example, but not by way of limitation), periodontal disease, Alzheimer's disease, cardiovascular disease, arthritis, adverse pregnancy outcomes, and the like.

The diagnostic microarray device may be formed in any manner and of any materials that allow the device to function in the manner described or otherwise contemplated herein. Methods of forming such microarray devices are known in the art and commercially available. Non-limiting examples of device platforms that can be utilized in accordance with the present disclosure include Axiom Biobank Genotyping Arrays (ThermoFisher Scientific, Waltham, MA), Illumina Microarrays (Illumina, Inc., San Diego, CA), TaqMan® Arrays (ThermoFisher Scientific, Waltham, MA), and the like. These types of arrays are typically produced to determine a genotype and are often genome wide or restricted to only one or two genes to help identify known genetic diseases. The present disclosure utilizes these microarray device platforms in a new and unique manner, by restricting the array to a group of up to about eight specific genotypes to detect a specific disease-associated phenotype.

The substrate of the diagnostic microarray device may be formed of any glass material that will allow the device to function as described herein. In a particular (but non-limiting) embodiment, the substrate is formed of a glass ionomer material. The various probes utilized are then formed on or otherwise attached to/associated with the glass material.

The various microarray devices known in the art detect binding of a nucleic acid molecule from a biological sample to each of the probes in different manners. For example (but not by way of limitation), nucleotide sequences may be isolated from the biological sample (and optionally treated to one or more fragmentation, amplification, and/or cDNA/cRNA preparation steps) and then labeled (such as, but not limited to, with a fluorescent dye) prior to passing the sample over the microarray device; in this manner, binding/hybridization of a labeled target nucleic acid to a probe of the device yields a positive result for the target allele to which the probe is directed. In another alternative, fragmented nucleotide sequences isolated from the biological sample are subjected to one or more amplification and/or cDNA/cRNA preparation steps that utilize labeled nucleotides in the preparation of the amplification/cDNA/cRNA products. For example (but not by way of limitation), one or more biotinylated nucleotides may be utilized in the sample preparation steps, and binding/hybridization of a target allele to a probe of the microarray device can be detected using a biotin binding agent (i.e., avidin, streptavidin, etc.); alternatively, one or more fluorescent dyes may be utilized in place of the biotin to label the nucleotides (i.e., up to four fluorescent dyes may be utilized, one for each nucleotide). In another alternative, fragmented nucleotide sequences isolated from the biological sample are allowed to first hybridize to a probe of the microarray device, and then labeled nucleotides (wherein each nucleotide is labeled with a different fluorescent dye or other label) are added to extend any single stranded portion of the hybridized complex.

In addition, each of the probes may be modified in any manner described herein or otherwise known in the art that will further aid in detection of the particular alleles/SNPs. For example, but not by way of limitation, each of the probes may have an enhancer of hybridization and/or fluorescence attached thereto.

One particular (but non-limiting) embodiment of the microarray devices utilizes the TaqMan© genotyping system. TaqMan® genotyping assays, considered the gold standard for allele detection, consist of pre-optimized PCR primer forward and reverse pairs and two probes for allelic discrimination, one to detect the allele (for example G or A) in forward sequence, and the other (C or T) in reverse sequence. The two TaqMan® probes have different fluorescent dyes at the 5′ ends (for example, but not by way of limitation, one probe with a fluorescein derivative (FAM) dye and the other with a different fluorescent dye (VIC) at the 5′ end). On the 3′ end of both probes are minor groove binders (MGB) and nonfluorescent quenchers (NFQ). These assays are used to amplify and detect specific alleles in genomic DNA (gDNA). Genomic DNA is introduced into a reaction mixture consisting of TaqMan® Genotyping Master Mix, the forward and reverse primers and the two TaqMan® MGB Probes. Each TaqMan® MGB Probe anneals specifically to a complementary sequence, if present, between the forward and reverse primer sites. When the probe is intact, the proximity of the quencher dye to the reporter dye suppresses the reporter fluorescence. The exonuclease activity of AmpliTaq Gold® DNA Polymerase (ThermoFisher Scientific, Waltham, MA) cleaves only probes hybridized to the target. Cleavage separates the reporter dye from the quencher dye, increasing fluorescence by the reporter. The increase in fluorescence occurs only if the amplified target sequence is complementary to the probe. Thus, the FAM or VIC fluorescence signal generated by PCR amplification indicates which alleles are in the sample.

Certain non-limiting embodiments of the present disclosure are directed to kits for performing any of the methods disclosed or otherwise contemplated herein. The kits include one or more of any of the diagnostic microarray devices disclosed or otherwise contemplated herein. The kits may further include one or more of any reagents utilized in sample preparation and/or conductance of the assay. For example, but not by way of limitation, the kits may include one or more reagents utilized in the fragmentation of the DNA of the biological sample, amplification of the DNA of the biological sample, preparation of cDNA/cRNA from the biological sample, fluorescent dyes for attachment to the fragmented nucleotide sequences isolated from the biological sample, labeled nucleotides (such as, but not limited to, biotinylated nucleotides or fluorescently- or otherwise labeled nucleotides), and the like, as well as any combinations thereof.

In addition to the diagnostic microarray device(s) and sample preparation/assay reagent(s) disclosed herein above, the kits disclosed herein may further contain any other component(s) or reagent(s) for use when conducting any of the particular assays described or otherwise contemplated herein. For example (but not by way of limitation), the kit may further include positive and/or negative control reagents, wash solutions, etc. The nature of additional reagent(s) present in the kits will depend upon the particular microassay format, and identification thereof is well within the skill of one of ordinary skill in the art; therefore, no further description thereof is deemed necessary.

Also, the compositions/reagents present in the kits may each be in separate containers/compartments, or various compositions/reagents can be combined in one or more containers/compartments, depending on the reactivity and stability of the compositions/reagents. In addition, the kit may further include a set of written instructions explaining how to use the kit. A kit of this nature can be used in any of the methods described or otherwise contemplated herein.

Certain non-limiting embodiments of the present disclosure are directed to method of treating or reducing the occurrence of at least one condition/disease in a subject, wherein the at least one condition/disease is selected from periodontal disease, Alzheimer's disease, cardiovascular disease, arthritis, adverse pregnancy outcomes, and the like. The method includes the steps of: contacting any of the diagnostic microarray devices disclosed or otherwise contemplated herein with at least a portion of a biological sample from the subject and incubating the diagnostic microarray device under conditions that allow for detection of nucleic acid bound to any of the probes associated with the substrate of the diagnostic microarray device; determining the alleles present for each of the SNPs IL1A-889, IL1B+3877, IL1B-511, IL6-1363, IL10-597, IL10-1082, CD14-260, and/or COX2+8473 to define a genotype for the biological sample; and administering at least one therapeutic agent and/or recommending at least one therapeutic protocol to the subject when: (i) the genotype for the biological sample comprises IL1B+3877CT and IL1B-511GG; (ii) the genotype for the biological sample comprises IL1B+3877CT and IL1B-511GA, and wherein the genotype does not contain any of the following alleles: IL6-1363T, IL10-1082C, and/or COX2+8473G; or (iii) the genotype for the biological sample comprises IL1B+3877CC, IL1B-511GA, and IL6-1363GT. In addition, the presence in the genotypes of (i), (ii), and/or (iii) of one or more of IL1A-889(GA), IL1A-889(AA), IL10-597(TT), CD14-260(AA), and/or COX2+8473(AG) can indicate severe disease, early tooth loss, and increased susceptibility to periodontitis-associated diseases and conditions, such as (but not limited to) Alzheimer's disease, cardiovascular disease, arthritis, adverse pregnancy outcomes, and the like.

The methods of the present disclosure may include any additional steps necessary for isolation and/or preparation of the biological sample prior to contact with the diagnostic microarray device. Non-limiting examples of additional steps that can be performed before contacting the biological sample with the diagnostic microarray include isolating nucleotide sequences from the biological sample; treating the isolated nucleotide sequences to one or more amplification and/orcDNA/cRNA preparation steps (with or without labeled nucleotides); and/or labeling the isolated nucleotide sequences. In addition, the incubating and determining steps may involve one or more particular (but non-limiting) steps specific for a particular microarray platform, as described in detail herein above.

Any therapeutic agent(s) effective in reducing the occurrence and/or severity of periodontitis may be utilized as the at least one therapeutic agent administered in accordance with the methods of the present disclosure. Non-limiting examples of therapeutic agents that may be utilized in accordance with the present disclosure include a furzinc oxide/zinc citrate toothpaste, a stannous fluoride toothpaste, an antimicrobial rinse, an oral antibiotic, an antibiotic gel, antibiotic microspheres, an antiseptic chip, Atuzaginstat (COR388), and the like, as well as any combinations thereof. Particular (but non-limiting) examples of antibiotics, antimicrobials, and antiseptics that may be utilized in accordance with the present disclosure include doxycycline, minocycline, chlorhexidine, carbamide peroxide, and the like, as well as any combinations thereof.

The therapeutic protocol(s) utilized in accordance with the present disclosure include any therapeutic protocols effective in reducing the occurrence and/or severity of periodontitis. Non-limiting examples thereof include performing dental scaling and root planing at an increased rate (i.e., increasing professional cleanings from once every six months to at least about once every month, every two months, every three months, etc.), as well as periodontal surgical options known in the art.

### Example

An Example is provided hereinbelow. However, the present disclosure is to be understood to not be limited in its application to the specific experimentation, results, and laboratory procedures disclosed herein. Rather, the Example is simply provided as one of various embodiments and are meant to be exemplary, not exhaustive.

### SUMMARY

Susceptibility to periodontal disease (periodontitis) is unpredictable. This common, chronic inflammatory disease begins when microbial biofilms adhere to teeth at gingival crevices and activate innate immunity to bacteria when oral hygiene stops. Gingival crevicular fluid (GCF), an inflammatory exudate, develops due to biofilm lysine decarboxylase impairing the dental attachment by depleting basal epithelial cells of lysine. As described herein after, a curve of GCF/min against biofilm lysine concentration after a week of no oral hygiene was interpreted to show two parallel curves, strong or weak GCF responses. On examining alleles of nine genes associated with periodontitis, a correct separation of 60% of participants was obtained by the A allele of IL1B-511 or the T allele of IL1B+3877, with the proviso that if both IL1B-511(A) and IL1B+3877(T), a conflicted genotype, were present, epistasis occurred, and homozygous CD14-260(A) or homozygous IL10-597(T) assigned weak response, and homozygous IL10-597(G) or a heterozygous COX2-8473(A) assigned strong response. In two other individuals, the T allele of IL6-1363 was epistatic; it changed the phenotype of IL1B+3877(T) from weak to strong in a fifth host with the conflicted genotype, and that of homozygous IL1B+3877(C) from strong to weak (epistasis) in host 11. Together, these genotypes correctly identify strong or weak innate immune responses to microbial biofilms in all 15 of the participants available to provide a DNA sample.

It has long been recognized that cleaning teeth prevents gingival inflammation (gingivitis), but what has not been recognized is that the initial development of gingivitis activates innate immunity to bacteria. Innate immunity is a genetically controlled system of events in response to oral bacteria or any bacterial infection, but in the case of gingival inflammation (gingivitis), the initial host response was found to be caused by a complex mixture of bacteria from saliva. The weak innate immunity response phenotype identified herein increases susceptibility to periodontitis and other associated chronic diseases such as (but not limited to) Alzheimer's diseases.

### Introduction:

Current therapy for preventing periodontal disease is primarily self-administered oral care (tooth brushing twice daily), supplemented with professionally administered care twice annually to remove precipitated calcium deposits (dental calculus), along with ensuring no smoking and controlling type 2 diabetes. The overarching need for lifelong oral hygiene indicates the presence in dentally attached biofilms of bacterial agents that induce gingival inflammation. The bacterial enzyme lysine decarboxylase from E. corrodens (LdcE) was identified as one of these agents (Lohinai, et al., J Periodontol. (2015) 86(10):1176-1184; and Lohinai, et al., J Periodontol. (2012) 83(8):1048-56). Other studies indicate that antibodies to this enzyme retard gingivitis development in the beagle dog model (Peters, et al., Vaccine (2012) 30(47):6706-12), and that its inhibition by zinc ions explains the well-known efficacy of zinc toothpastes in controlling human periodontal disease (Levine, et al., J Dent (2021) 104:103533). Unlike acid-induced lysine decarboxylases secreted from Escherichia coli and other gastro-intestinal bacteria (Ldcl), LdcE is constitutively present on the E. corrodens outer surface and is active in alkali in addition to dilute acid. The cadaverine produced by LdcE also contributes to changes in the microbiome of gingivodental biofilms and enhances growth on GCF instead of saliva within the gingival crevice.

The initiation of inflammation and its exacerbation by bacteria that adhere to teeth is called innate immunity. Innate immunity is an initial, non-specific inflammatory response to an internal or external injury or foreign invader. Persistence of innate immunity induces acquired immunity, a highly specific response to its foreign antigens. Host products of masticatory damage (DAMPs) and related microbial or pathogen activated microbial products (MAMPs or PAMPs) bind to pattern recognition receptors, PRRs. PRRs are mostly Toll-like receptors (TLRs) on the outer surface of epithelial cells such as the junctional epithelial attachment (JE), or nucleotide oligomerization domain receptors (NLRs) in the JE cytosol. Once bound, the DAMP-, MAMP-, or PAMP-PRR complexes activate alarmins such as IL6 and IL1A that then activate other interleukins and cytokines to induce innate and adaptive immunity. The activated IL1 production of cytokines and chemokines attracts neutrophils that remove the MAMPs and DAMPs and repair the damage. Yet once calculus has developed, calcified biofilms remain dentally attached or redevelop despite professional care, and periodontitis continues to progress.

IL1 is composed of two genes, IL1A which is constitutively expressed as IL-1α, a protein already activated to induce inflammation following MAMP binding by PRRs on the JE cell surface or within an epithelial cell cytosol. IL-1α induces the expression of gene IL1B and activates its protein (IL-1β). The amount of IL-1β is strongly associated with gingival and periodontal inflammation (Levine and Lohinai, J Clin Med (2021) 10(11); and Chapple, et al., J Clin Periodontol. (2015) 42 Suppl 16:S71-S76). Subsequently cleavage of the IL-1(3 protein by an extracellularserine protease, granzyme B, enhances IL-1β proinflammatory cytokine production (Afonina, et al., Mol Cell (2011) 44(2):265-78). During three weeks of EG, IL-1α is secreted first, but IL-1β expression catches up and then increases further as gingivitis persists and periodontitis develops (Tsalikis, et al., J. Int. Acad. Periodontol. (2002) 4(1):5-11; Offenbacher, et al., J Clin. Periodontol. (2010) 37(4):324-333). Therapy and the re-institution of oral hygiene downregulate IL-1β expression, and the removal of MAMPs reduces IL-1α activity (Kaushik and Cuervo, Nat Rev Mol Cell Biol (2018) 19(6):365-381). Nevertheless, mastication maintains enough DAMPs to maintain a low-level activation of IL-6 (Dutzan, et al., Immunity. (2017) 46(1):133-147), and oral hygiene keeps the epithelial attachment intact by preventing LdcE from removing lysine.

Because the division of microbial response to LdcE activity in EG and in periodontitis is significant, this Example investigated whether strong and weak inflammatory responses were genetically determined phenotypes after a week of EG in a population of healthy young adults. The strong (fast) or weak (slow) phenotype can provide a basis for deciding who requires drugs that prevent severe periodontal disease and its associated systemic chronic infections, such as (but not limited to) Alzheimer's disease, cardiovascular disease, and the like. The results presented in this Example demonstrate that an increased susceptibility to tooth loss should predominate in the weak phenotype.

### Materials and Methods:

The EG procedures utilized in this Example are outlined in Lohinai et al. (2012).

The human protocol described below was reviewed and approved by the Ethics Committee of the Hungarian Medical Research Council (Approval no. 11878-1/2006-1017EKL), and Semmelweis University, Budapest, Hungary (Approval no. TUKEB 140/2006) as reported previously (Lohinai, et al., J Periodontol. (2012) 83(8):1048-56). At a follow-up visit, 15 of the 16 individuals who had participated in the prior EG study agreed to provide buccal cheek scrapings from which genomic DNA was purified by column separation. The 16th individual had emigrated from Hungary and was unavailable. SNP genotyping analysis used the TaqMan® assay method described by ThermoFisher Scientific (Waltham, Mass., USA). Each assay kit contained two probes, one in which the SNP of interest was present and one in which it was absent. One end of each probe had a covalently attached fluorescent molecule, and the other end an attached enhancer of both hybridization and fluorescence. Fluorescence measurements during genomic DNA amplification in the presence of both probes were graphed over time. From this graph, the intensity of fluorescence indicated whether the SNP was present in all of a host's genomic DNA, or in half or none of the DNA.

The SNP variants (alleles) were from nine genes whose alleles were reported to influence periodontitis: IL1A-889A (Karimbux, et al., J Periodontol. (2012) 83(11):1407-1419; Giannobile, et al., J Dent. Res. (2013) 92(8):694-701; Shirodaria, et al., J Dent. Res (2000) 79(11):1864-1869), IL1B+3877T (Wu, X., et al., J. Periodontal Res (2015) 50(1):52-61), IL1B-511 (Rogus, et al., Hum Genet (2008) 123(4):387-98), IL6-1363 (Nibali, et al., Journal of Clinical Periodontology (2008) 35(3):193-8), IL10-597 (Li, et al., Dis Markers (2018) 2018:2645963), IL10-1082 (Wong, et al., Innate Immun (2009) 15(2):121-8), CD14-260, COX2+8473 (Prakash, et al., Oral Dis (2015) 21(1):38-45; Lohinai, et al., Life Sci (2001) 70(3):279-90), and MMP8-799 (lzakovicova Holla, et al., Archives of Oral Biology (2012) 57(2):188-96). These genes are listed in Table 1 along with their Reference SNP (rs) numbers and MAFs. Each SNP is numbered by the position of the SNP-altered nucleotide before (minus) or after (plus) the first protein coding nucleotide. Significant associations of strong or weak phenotype with genotype were determined using Fisher's Exact Test (Table 2).

TABLE 1
Genes Reported to Associate with Periodontitis

| Gene name and site^{a} | rs number^{b} | MAFα Euro^{c} |
|---|---|---|
| IL1A − 889(A) | rs1800587 | 0.285 |
| IL1B + 3954(T) | rs1143634 | 0.237 |
| IL1B − 511(A) | rs16944 | 0.335 |
| IL6 − 1363(T) | rs2069827 | 0.085 |
| IL10 − 597(T) | rs1800872 | 0.085 |
| CD14 − 260(A) | rs2569190 | 0.485^{c} |
| COX2 + 8473(A) | rs5275 | 0.337 |
| IL10 − 1082(T) | rs1800896 | 0.473^{c} |
| MMP8 − 799(G) | rs11225395 | 0.464^{c} |

Gene name and location of the allelic site are indicated by number of nucleotides before (+), or after (−) the translation start site, and the minor allele nucleotide (brackets).
References for association with periodontal disease are provided in the text.
^{a}Single nucleotide (SNP) reference cluster (rs) ID numbers for each periodontitis-associated gene.
^{b}Minor allele frequency (MAF), validated in a large European population (22).
^{c}Codominant.

TABLE 2
IL1B Alleles at −511 and +3194, Respectively,
Indicate Strong or Weak Phenotypes

| IL1B + 511 | No A | Wi A |
|---|---|---|
| Strong | 0 | 9 |
| Weak | 3 | 3 |
| Fisher exact test statistic = 0.044, p < 0.05. | | |
| IL1B + 3954 | No T or Ep^{a} | Wi T or Ep |
| Strong | 7 | 2 |
| Weak | 0 | 6 |

Fisher exact test statistic = 0.007, p < 0.01.
^{a}T allele or CC allele with epistatic (Ep) IL6 − 1363T in the genome (original).

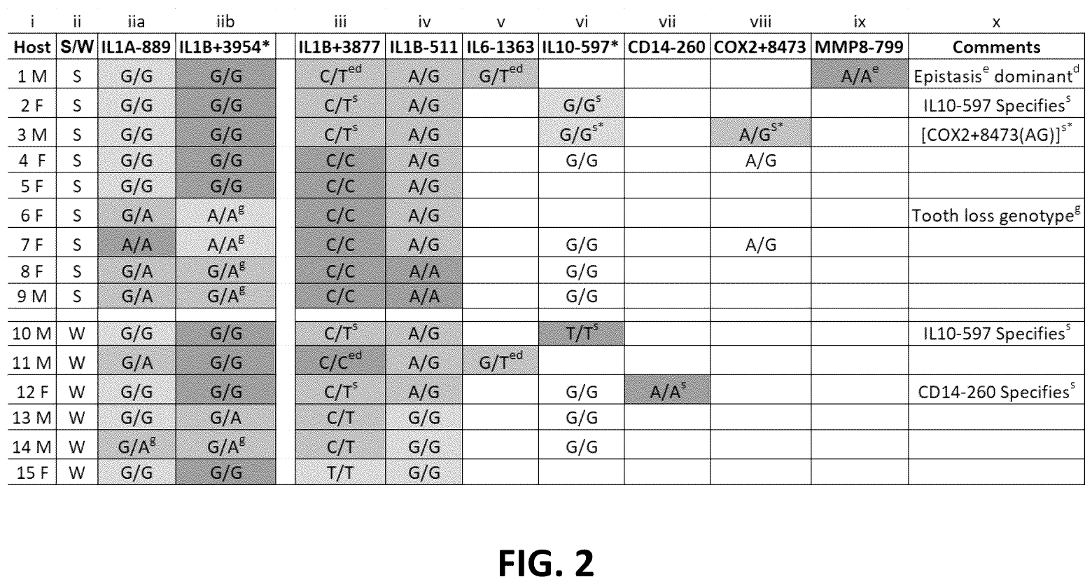

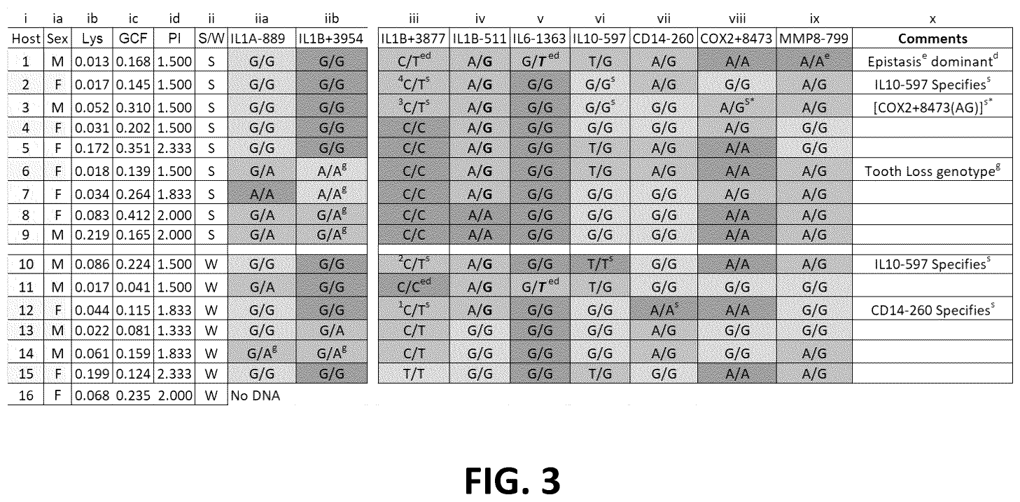

In FIGS. 2 and 3, each participant is listed by ID number, their strong or weak response, and the presence or absence of selected alleles from each of the nine genes. A medium gray background indicates that the SNP was present in only one of the two DNA chromosomes (heterozygous), a dark gray background indicates that the reference SNP allele was present on both chromosomes (homozygous reference SNP), and a light background indicates that the alternate SNP was present on both chromosomes (homozygous SNP positive). If the gene was read from the complimentary chromosome, the homozygous reference SNP allele is indicated with a light gray background, and chromosomes possessing only the alternate SNP are indicated with a dark gray background. In FIG. 2, the columns show the ID number and sex of each participant, whether their GCF response was strong or weak, and the alleles of all genes except IL10. Data not relevant to the findings was omitted to simplify understanding. The complete data set is present in FIG. 3, in which additional Columns ia, ib, iia, and xi indicate lysine contents, GCF exudation rates, PI values, and MMP8-799 gene alleles, respectively.

Statistical analysis: Using GCF exudation rate as the outcome, strong and weak GCF responses were examined using a quadratic polynomial regression model using the JMP Pro 13.1 computer program (SAS Institute Inc., Cary, NC.). Significance of the GCF response (main effect), and its interactions with lysine content and lysine content-squared (independent variables) were determined using an F-ratio test with 3 degrees of freedom to compare the sum of squares explained by the two independent terms (Lys and Lys-squared) relative to the sum of squares error. Differentiation of strong and weak responses by genes and genotypes in Table 2 was initially determined using the Fisher exact statistic (Social Science Statistics website).

### Results:

In the population of 15 Hungarian young adults, the occurrence of five hosts with the tooth loss genotype, IL1B+3954(A) plus IL1B-511(A), was 33% which approximated that predicted from their minor allele frequencies (MAFs) in a large population (Karimbux, et al., J Periodontol. (2012) 83(11):1407-1419). The preference of IL1B-511(A) for strong response was significant (Table 2). The (T) allele of IL1B+3877 for weak response was not significant unless the T allele of IL6-1363 was also present. This allele changed the phenotype of a homozygous normal IL1B allele, IL1B+3877(CC), from strong to weak, and that of a IL1B+3877(CT) allele from weak to strong, a genetic event called epistasis (Wei, et al., Nat Rev Genet (2014) 15(11):722-33). Because the IL1B+3877 phenotype was subordinate to IL6-1363(T) in the genome, it is referred to as the hypostatic gene and made the association of IL1B+3877 with weak phenotype significant (Table 2). The epistatic/hypostatic activity is dominant, and identified in FIGS. 2 and 3 by a superscript ‘ed.’

The current results are shown in FIG. 2, and a more complete rendition in FIG. 3. Table 3 shows the results: 1) 6 strong responders, the genotype of which is the reference SNP at IL1B+3877(CC) plus the alternative SNP at IL1B-511(A); 2) 3 weak responders the genotype of which is the alternative SNP at IL1B+3877(T) and the reference SNP at IL1B-511(GG). Two hosts exhibited epistasis by the alternate T allele of IL6-1363: a 7^{th} host with [IL1B+3877(CC) plus IL1B-511(A)] was converted to weak, and a second host with a conflicted phenotype [(IL1B+3877(T) plus IL1B-511(A)] was converted to strong. There remained four hosts whose genotype was also conflicted, IL1B+3877(CT), potentially weak, and IL1B-511(GA), potentially strong (Table 2).

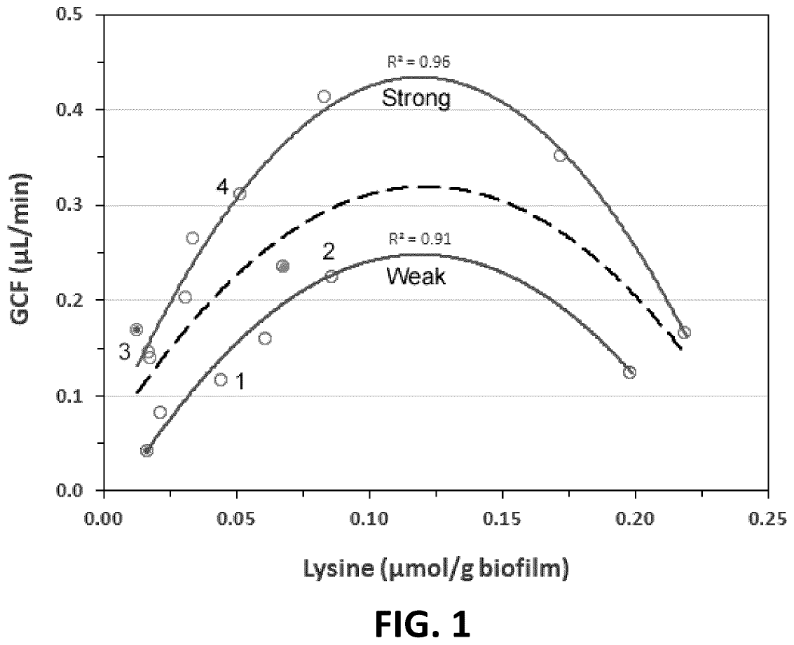

FIG. 1 shows the relationship of genotype to strong and weak GCF response graphically by adding a number to indicate their respective positions on the graph. Two of these hosts exhibited a weak phenotype in the presence of the homozygous (A) allele of CD14-260¹ or the homozygous (T) allele of IL10-597². The other two exhibited a strong phenotype if the second gene was the heterozygous (G) allele of COX2-8473(AG)³, or the major (C) homozygous allele of IL10-597⁴. FIG. 1 also indicates that the two individuals who possessed the epistatic IL6-1363(T^{e}) gene had the two smallest biofilm lysine contents of all 15 participants in this Example. The most strong GCF responders were identified by the presence of the A allele of IL1B-511 along with the homozygous common (C) allele of L1B+3877 (six responders), and weak GCF responders by the T allele of IL1B+3877 along with presence of the homozygous common (G) allele of IL1B-511 (three responders). In addition, one weak and one strong responder were determined by epistasis of IL1B+3877(CC) and (CT) by the T allele of IL6-1363, and the remaining four, who exhibited both IL1B-511A and IL1B+3877T, were resolved as strong or weak responders with unique third alleles as indicated in Table 3.

TABLE 3
Summary of Genotypes That Detect Strong and Weak Phenotypes

| Host | Phenotype | Genotype | | |
|---|---|---|---|---|
| 1M | S | IL1B + 3954CT^{e} | IL1B − 511GA | IL6 − 1363GT^{e} |
| 2F | S | IL1B + 3954CT | IL1B − 511GA | IL10 − 597CC^{s} |
| 3M | S | IL1B + 3954CT | IL1B − 511GA | COX2 + 8473AG^{s}* |
| 4FM | S | IL1B + 3954CC | IL1B − 511GA | |
| 5F | S | IL1B + 3954CC | IL1B − 511GA | |
| 6F | S | IL1B + 3954CC | IL1B − 511GA | |
| 7F | S | IL1B + 3954CC | IL1B − 511GA | |
| 8F | S | IL1B + 3954CC | IL1B − 511AA | |
| 9M | S | IL1B + 3954CC | IL1B − 511AA | |
| 10M | W | IL1B + 3954CT | IL1B − 511GA | IL10 − 597TT^{s} |
| 11M | W | IL1B + 3954CC^{e} | IL1B − 511GA | IL6 − 1363GT^{e} |
| 12F | W | IL1B + 3954CT | IL1B − 511GA | CD14 − 260AA^{s} |
| 13M | W | IL1B + 3954CT | IL1B − 511GG | |
| 14M | W | IL1B + 3954CT | IL1B − 511GG | |
| 15F | W | IL1B + 3954CT | IL1B − 511GG | |

Bold font indicates individuals whose phenotype depends only on alleles of IL1B + 3945 and IL1B − 511.
Underlining indicates individuals whose phenotype depends on epistasis of IL1B + 3954 alleles by IL6 − 1363T.
No highlighting indicates individuals whose phenotype depends on a third gene other than IL6 − 1363GT
Third genes with superscript ‘s’ can make IL1B − 511GA dominant (strong phenotype), or IL1B + 3877CT dominant (weak phenotype).
The superscript s* indicates an alternative 3rd gene to IL10 − 597CC for host 3.

GCF responses to biofilm lysine are therefore strong or weak, and governed by genotypes that are mostly based on one of two alleles of IL1B. The site located at IL1B-511 upstream of the encoded protein transcription site is protective, and that of IL1B+3877 well downstream is destructive except in presence of IL6-1363(T), in which the destructive and protective alleles of IL1B are reversed. These three genes indicate the strong or weak susceptibility to moderate and severe periodontitis of our Hungarian population with 73% accuracy. The various other third SNP alleles of genes identified in FIG. 2 increase the prediction to 100% accuracy, but additional third alleles could be present in a larger population.

### Significance:

As noted above, the GCF response to biofilm lysine measures the strength of innate immunity to a mixture of bacteria as two parallel curves. Nine of the 16 tested participants possessed an innate immune response that was strong (upper curve), and seven participants possessed a response that was weak (lower curve). Fifteen of these individuals agreed to provide a sample of their DNA, but the 16th had emigrated and was unavailable. A strong response was associated with the SNP IL1B-511(A), and a weak response with IL1B+3877(T), unless IL6-1363(T) was present, and changed the response of IL1B+3877(T) to be strong and that of its absence to be weak. Others have indicated that IL1B+3877(T) and IL6-1363(T) indicate moderate to severe periodontitis, greater gingival inflammation, and bacterial dysbiosis (i.e., presence of the ‘red’ complex) in periodontal pockets (Wu, X., et al., J. Periodontal Res (2015) 50(1):52-61) (Nibali, et al., J Clin Periodontol (2008) 35(3):193-198). The remaining four individuals were characterized as strong or weak by the presence of a third gene in the absence of IL6-1363(T).

Although the development of gingivitis during EG is thought to lead to periodontitis, observing that transition would cause an irreversible loss of periodontal attachment, making the experiment unethical. Nevertheless, the strong outward flow of GCF removes particles and bacteria from deepened crevices called periodontal pockets, suggesting less incubation time for red complex and periodontitis development. Likewise, the inhibition of GCF exudation by repeated tobacco smoking results in more incubation time for ‘red’ bacterial complex development, and a 5-fold greater likelihood of developing periodontitis. Indeed, smoking is a major risk factor for periodontitis and may be responsible for more than half of periodontitis cases among adults in the United States for at least the last 30 years (Tomar and Asma, J Periodontol (2000) 71(5):743-51; Leite et al., Am J Prev Med (2018) 54(6):831-841). That a strong GCF response to gingivitis protects from dysbiosis, and a weak GCF response enhances dysbiosis, is supported in vitro (Naginyte et al., Sci Rep (2019) 9(1):5491-5500). As noted above, a dysbiotic ‘red’ bacterial complex grows out from salivary bacteria in an in vitro fluid resembling GCF after the growth medium is changed twice weekly for three weeks. Thus, changing the medium more frequently would retard ‘red’ complex development and periodontitis. The rate of GCF exudation in FIG. 1 was about twice as great in strong responders than in weak responders. Therefore, the presence of genotypes associated with strong GCF exudation indicate that periodontitis and its related diseases should be controlled more easily than those with weak GCF exudation.

Others have reported statistically coherent subgroups of gingivitis development during EG, 36% fast and 64% slow (Nascimento, et al., European Journal of Oral Sciences (2019) 127(1):33-39) or 42% fast, 28% intermediate, and 28% slow (Bamashmous, et al., PNAS USA (2021) 118(27)). Compared with the 56-60% of strong responders in this Example, the separation of strong from weak responders in these other studies appears incomplete, although saliva or GCF in fast responders exhibited greater levels of other pro-inflammatory cytokines than slow responders in both studies (Nascimento, et al., Cytokine (2019) 115:135-141; Silbereisen, et al., JDR Clinical and Translational Research (2019) 2380084419844937; Bamashmous, et al., PNAS USA (2021) 118(27)). It was concluded that the use of biofilm accumulation (PI) instead of biofilm lysine identified only the strongest and fastest of the GCF responders. Also, the use of clinical gingivitis to measure inflammation requires at least two weeks of EG, whereas GCF exudation can measure inflammation accurately at one week (Trombelli et al., 2004 J Clin Periodontol(2004) 31(4):239-252).

It has been noted herein that there are two levels of response to periodontitis, with a slightly smaller fraction of the population developing more disease. In another study, middle-aged, non-smoker patients from the University of Connecticut with periodontitis were enrolled between 2010 and 2012 (Hong, et al., PloS one (2015) 10(5):e0127077; Hong, et al., PloS one (2016) 11(2):e0148893). Nineteen had few deep pockets (crevices<5 mm deep) and a microbiome cluster, in which P. gingivalis accounted for about 12% of the biofilm bacteria. The remaining 15 participants (44% of the total) exhibited significantly more periodontal inflammation, deeper pockets (crevices>5 mm), and dysbiotic biofilms, in which P. gingivalis accounted for 56% of the bacteria along with greater amounts of the accompanying red complex, and therefore the weak GCF responder phenotype (hong t al., PLoS One 2015, 10(5):1-14). The 44% of patients possessing the cluster-enriched red bacteria have also been indicated by the presence of the IL1B+3954(GA) genotype (Pani, et al., J Periodontal Res (2021) 56(3):501-511).

Application to periodontal and Alzheimer's disease susceptibility: Taken together, the results demonstrate that moderate to severe periodontitis is caused by weak innate immunity that enhances susceptibility to biofilms possessing P. gingivalis, the keystone bacterium of the red microbial complex. There are currently no reports of two different levels of innate immunity to an apparently similar salivary microbiome in humans; for example, see Diaz et al., 2015 (Diaz, et al., J Calif. Dent. Assoc. (2016) 44(7):421-435). As already noted, P. gingivalis is present in the brain of more than 90% of Alzheimer's patients after autopsy (Dominy, et al., Sci Adv. (2019) 5(1):eaau3333), and patients exhibiting moderate periodontal disease for 10 years have a nearly two-fold greater risk of developing Alzheimer's disease; however, whether periodontal disease is the cause or effect of cognitive decline on oral hygiene is still uncertain (Chen, et al., Alzheimers Res. Ther. (2017) 9(1):56).

This Example reported that strong or weak innate immunity to the initial accumulation of biofilm in 73% of young adults is genetically determined by different alleles of IL1B alone or by epistasis in the presence of IL6-1363(GT). The remainder possess contradictory alleles of IL1B resolved by a third allele which differs in different individuals. IL1B+3877(CT), associated here with weak response, also associates with a greater presence of red complex bacteria (Pani, et al., J Periodontal Res (2021) 56(3):501-511) and greater levels of P. gingivalis in the oral cavity. Therefore, based on the report that all patients exhibiting moderate periodontal disease for 10 years have a nearly two-fold greater risk of Alzheimer's disease, separating them into strong and weak responders indicates a four to five-fold greater risk for weak responders.

While not wishing to be bound by a particular theory, once periodontal pathogens develop in dental biofilms and enough P. gingivalis is present, these bacteria might enter the bloodstream through a periodontal pocket (deepened crevice) whose epithelial barrier is inflamed and compromised by gingipains, powerful proteases that may eventually cause a gingipain-mediated brain infection. The ensuing disruption of equilibrium between pro- and anti-inflammatory mediators within the brain likely results in chronic neuroinflammation that eventually promotes Alzheimer's disease. A similar situation is caused by mutations of genes encoding immune receptors, especially the triggering receptor expressed on myeloid cells type 2 (TREM2). TREM2 deficiency reduces clustering of the microglia that digest the plaques associated with Alzheimer's disease development. These plaques are accumulated protein fragments between neurons. If not continuously removed, they disrupt the brain's normal disposal process, and eventually impact cognition.

The most common TREM2 mutation is an amino acid sequence change in which arginine encoded at amino acid 47 is changed to a histidine residue (R47H). The change in protein sequence associates with reduced microglial function and faster AD development. Likewise, AD mice heterozygous for TREM2 R47H exhibit fewer immune cells and more neuron degradation (Liccardo, et al., Front. Physiol. (2020) 11:683). Alternatively, excess protein fragments in the brain from gingipain Kgp and Rgp proteinases could mimic a TREM2 R47H mutant with the same over-accumulation of protein fragments. These and other substantial genetic and functional findings point to a central role for weak innate immunity in neurodegenerative diseases (Nara, et al., Journal of Alzheimer's Disease: JAD (2021) 82(4):1417-1450; Shi, et al., Nat. Rev. Immunol. (2018) 18(12):759-772), and are therefore relevant to this disclosure. Therefore, the present disclosure includes the addition of TREM2 variants such as (but not limited to) R47H to the devices, kits, and methods of the present disclosure to better account for Alzheimer's disease risk.

Atuzaginstat (COR388) is an orally administered brain-penetrating small molecule gingipain inhibitor, and a potentially new drug candidate that inhibits Kgp action in vitro and in mice (Dominy, et al., Sci Adv. (2019) 5(1):eaau3333). In a study conducted by Cortexzyme (San Francisco, CA), the efficacy of Atuzaginstat on clinical endpoints of periodontitis was measured in 233 volunteers. According to their disclosure, antibody measurements were expected to exhibit biofilms that had a high content of P. gingivalis determined by quantitative antibody measurements. Unfortunately, this approach indicates immune responsiveness, not antigen load. Excessive amounts of antigen can cause immune-suppression, and weak innate immunity can prevent a strong antibody response from developing. Consequently, the weak innate immunity response genotypes disclosed herein could solve this problem. The genotypes could determine which participants in that study were likely to have small amounts of P. gingivalis in their biofilm (strong genotype) as well as those likely to have much larger amounts (weak genotype). As the latter group would be most likely to show an effect of the drug Atuzaginstat, it would determine clearly whether this or a related drug can provide significant protection from P. gingivitis periodontitis in adults 10-20 years prior to developing Alzheimer's disease.

### CONCLUSION

The percentage of the US population diagnosed with Alzheimer's dementia increases to 35% by age 85, similar to the fraction of those over age 30 with moderate to severe periodontal disease, and to the fraction of individuals at age 50 exhibiting excessive biofilm colonization with P. gingivalis. Thus, testing at least individuals of European descent for the genotypes described herein improves efforts to predict and prevent not only periodontal disease, but also Alzheimer's Disease, cardiovascular disease, and other periodontitis-associated diseases.

The two host genotypes described in this Example may explain why human biofilm microbiomes are expressed as two distinct clusters. One group's genotype is associated with allele IL1B-511(A). Their GCF is strong enough to remove bacteria from the gingival crevice and retard P. gingivalis-mediated periodontitis and Alzheimer's disease. The other group's genotype is associated with allele IL1B+3877(T) and epistasis of its non-T allele by IL6-1363(T). These alleles promote a weaker GCF exudate that lets more bacteria remain in the gingival crevice where they promote much more P. gingivalis colonization, and more disease (Hong, et al., PloS one (2015) 10(5):e0127077; Hong, et al., PloS one (2016) 11(2):e0148893).

Thus, in accordance with the present disclosure, there have been provided compounds, as well as methods of producing and using same, which fully satisfy the objectives and advantages set forth hereinabove. Although the present disclosure has been described in conjunction with the specific drawings, experimentation, results, and language set forth hereinabove, it is evident that many alternatives, modifications, and variations will be apparent to those skilled in the art. Accordingly, it is intended to embrace all such alternatives, modifications, and variations that fall within the spirit and broad scope of the present disclosure.

What is claimed is:

1. A diagnostic microarray device, comprising:
a substrate formed of a glass material;
at least sixteen probes attached or otherwise associated with the substrate, wherein each probe is associated with the substrate at a defined location for detection thereof, wherein each probe is specific to one allele of a single nucleotide polymorphism (SNP), and wherein the at least sixteen probes comprise:
a probe that detects a G allele of IL1A-889;
a probe that detects an A allele of IL1A-889;
a probe that detects a C allele of IL1B+3877;
a probe that detects a T allele of IL1B+3877;
a probe that detects a G allele of IL1B-511;
a probe that detects an A allele of IL1B-511;
a probe that detects a G allele of IL6-1363;
a probe that detects a T allele of IL6-1363;
a probe that detects a G allele of IL10-597;
a probe that detects a T allele of IL10-597;
a probe that detects a G allele of CD14-260;
a probe that detects an A allele of CD14-260;
a probe that detects an A allele of COX2+8473; and
a probe that detects a G allele of COX2+8473.

2. The diagnostic microarray device of claim 1, further defined as a diagnostic microarray device for detecting susceptibility to periodontal disease.

3. The diagnostic microarray device of claim 1, further defined as a diagnostic microarray device for detecting susceptibility to Alzheimer's disease.

4. The diagnostic microarray device of claim 1, further defined as a diagnostic microarray device for detecting susceptibility to cardiovascular disease.

5. The diagnostic microarray device of claim 1, further defined as a diagnostic microarray device for detecting susceptibility to arthritis.

6. The diagnostic microarray device of claim 1, further defined as a diagnostic microarray device for detecting susceptibility to adverse pregnancy outcomes.

7. The diagnostic microarray device of claim 1, wherein the substrate is formed of a glass ionomer material.

8. The diagnostic microarray device of claim 1, further defined as comprising at least eighteen probes, and wherein the at least eighteen probes further comprise at least two of:
a probe that detects an A allele of MMP8-799;
a probe that detects a G allele of MMP8-799;
a probe that detects a C allele of IL10-1082; and
a probe that detects a T allele of IL10-1082.

9. A method of treating or reducing the occurrence of periodontal disease in a subject, the method comprising the steps of:
contacting the diagnostic microarray device of claim 1 with at least a portion of a biological sample from the subject and incubating the diagnostic microarray device under conditions that allow for detection of nucleic acid bound to any of the probes associated with the substrate of the diagnostic microarray device;
determining the alleles present for each of the SNPs IL1A-889, IL1B+3877, IL1B-511, IL6-1363, IL10-597, CD14-260, and COX2+8473 to define a genotype for the biological sample;
administering at least one therapeutic agent and/or recommending at least one therapeutic protocol to the subject when:
(i) the genotype for the biological sample comprises IL1B+3877CT and IL1B-511GG;
(ii) the genotype for the biological sample comprises IL1B+3877CT and IL1B-511GA, and wherein the genotype does not contain any of the following alleles: IL6-1363T, and COX2+8473G; or
(iii) the genotype for the biological sample comprises IL1B+3877CC, IL1B-511GA, and IL6-1363GT.

10. The method of claim 9, wherein the at least one therapeutic agent comprises at least one of a furzinc oxide/zinc citrate toothpaste and a stannous fluoride toothpaste.

11. The method of claim 9, wherein the at least one therapeutic agent comprises Atuzaginstat (COR388).

12. The method of claim 9, wherein the at least one therapeutic protocol comprises performing dental scaling and root planing at least about once every three months.

13. A method of treating or reducing the occurrence of Alzheimer's disease in a subject, the method comprising the steps of:
contacting the diagnostic microarray device of claim 1 with a biological sample from the subject and incubating the diagnostic microarray device under conditions that allow for detection of nucleic acid bound to any of the probes;
determining the alleles present for each of the SNPs IL1A-889, IL1B+3877, IL1B-511, IL6-1363, IL10-597, CD14-260, COX2+8473 to define a genotype for the biological sample;
administering at least one therapeutic agent and/or recommending at least one therapeutic protocol to the subject when:
(i) the genotype for the biological sample comprises IL1B+3877CT and IL1B-511GG;
(ii) the genotype for the biological sample comprises IL1B+3877CT and IL1B-511GA, and wherein the genotype does not contain any of the following alleles: IL6-1363T, and COX2+8473G; or
(iii) the genotype for the biological sample comprises IL1B+3877CC, IL1B-511GA, and IL6-1363GT.

14. The method of claim 13, wherein the at least one therapeutic agent comprises at least one of a furzinc oxide/zinc citrate toothpaste and a stannous fluoride toothpaste.

15. The method of claim 13, wherein the at least one therapeutic agent comprises Atuzaginstat (COR388).

16. The method of claim 13, wherein the at least one therapeutic protocol comprises performing dental scaling and root planing at least about once every three months.

17. A method of treating or reducing the occurrence of at least one of cardiovascular disease, arthritis, and adverse pregnancy outcomes in a subject, the method comprising the steps of:
contacting the diagnostic microarray device of claim 1 with a biological sample from the subject and incubating the diagnostic microarray device under conditions that allow for detection of nucleic acid bound to any of the probes;
determining the alleles present for each of the SNPs IL1A-889, IL1B+3877, IL1B-511, IL6-1363, IL10-597, CD14-260, COX2+8473 to define a genotype for the biological sample;
administering at least one therapeutic agent and/or recommending at least one therapeutic protocol to the subject when:
(i) the genotype for the biological sample comprises IL1B+3877CT and IL1B-511GG;
(ii) the genotype for the biological sample comprises IL1B+3877CT and IL1B-511GA, and wherein the genotype does not contain any of the following alleles: IL6-1363T, and COX2+8473G; or
(iii) the genotype for the biological sample comprises IL1B+3877CC, IL1B-511GA, and IL6-1363GT.

18. The method of claim 17, wherein the at least one therapeutic agent comprises at least one of a furzinc oxide/zinc citrate toothpaste and a stannous fluoride toothpaste.

19. The method of claim 17, wherein the at least one therapeutic agent comprises Atuzaginstat (COR388).

20. The method of claim 17, wherein the at least one therapeutic protocol comprises performing dental scaling and root planing at least about once every three months.
